(19) United States
(12) Patent Application Publication
(10) Pub. No.: US 2024/0376285 A1
(43) Pub. Date: Nov. 14, 2024

(54) Method for Screening Solvent for Extracting Acrylonitrile Butadiene Styrene Copolymer, Method for Recycling Waste, Recycled Acrylonitrile Butadiene Styrene Copolymer, and Composition

(71) Applicant: LG Chem, Ltd., Seoul (KR)

(72) Inventors: Seungha Kim, Daejeon (KR); Eunbyeol Ko, Daejeon (KR); Kyoung Hoon Kim, Daejeon (KR); Hyung Sop Kim, Daejeon (KR); Gahyeon Lee, Daejeon (KR); Seung Yong Lee, Daejeon (KR); Yoonsun Jang, Daejeon (KR); Kyeong Hwan Han, Daejeon (KR)

(73) Assignee: LG Chem, Ltd., Seoul (KR)

(21) Appl. No.: 18/688,146

(22) PCT Filed: Sep. 2, 2022
(86) PCT No.: PCT/KR2022/013221

§ 371 Date: Feb. 29, 2024

(30) Foreign Application Priority Data
Sep. 3, 2021 (KR) 10-2021-0117879
Sep. 3, 2021 (KR) 10-2021-0117880
Sep. 1, 2022 (KR) 10-2022-0110818

(51) Int. Cl. C08J 11/08; C08L 55/02; G01N 33/44

(52) U.S. Cl. CPC C08J 11/08; C08L 55/02; G01N 33/442; C08J 2355/02; C08L 2207/20

(57) ABSTRACT

A method for screening a solvent for extracting acrylonitrile-butadiene-styrene (ABS) from waste using the following Equation 1:

ABS solubility score=x₁ Log₁₀Molecular Weight+x₂IAC_Mean

where Molecular Weight is the molecular weight of the solvent, IAC_Mean is the average information by an atomic number, and x₁ and x₂ are each a real number from 0 to 5. A method for recycling waste having ABS including screening the solvent using Equation 1 and extracting the ABS from the waste using the screened solvent is also provided. Additionally, a recycled acrylonitrile-butadiene-styrene copolymer and a recycled acrylonitrile-butadiene-styrene copolymer composition are provided.

### CROSS-REFERENCE TO RELATED APPLICATIONS

This application is a national phase entry under 35 U.S.C. § 371 of International Application No. PCT/KR2022/013221 filed on Sep. 2, 2022, which claims priority to Korean Patent Application Nos. 10-2021-0117879, 10-2021-0117880, and 10-2022-0110818 filed on Sep. 3, 2021, Sep. 3, 2021, and Sep. 1, 2022, respectively, the entire contents of which are incorporated herein by reference.

### TECHNICAL FIELD

The present specification relates to a method for screening a solvent for extracting an acrylonitrile-butadiene-styrene copolymer, a method for recycling waste, a recycled acrylonitrile-butadiene-styrene copolymer, and a recycled acrylonitrile-butadiene-styrene copolymer composition.

### BACKGROUND ART

Since plastic can be molded into various shapes by heat or pressure, and the quality of the material is getting better and better, things in everyday life are being replaced with plastic, and new things using the same are being developed.

Since plastics are convenient and easy to use, demand for products made of plastics, such as plastic bags, PET bottles, disposable items, packaging products, and electronic products, is gradually increasing all over the world.

Such used and discarded plastics are either incinerated or landfilled. In the case of incineration, many contaminants harmful to the human body are discharged, so it is preferable that the discarded plastics are landfilled rather than incinerated.

However, it takes a long time to naturally decompose most plastics after disposal, and the amount of plastics that end up in landfills is substantial, so interest in recycling plastics is increasing.

Among plastics, an acrylonitrile-butadiene-styrene copolymer (ABS) is widely used in battery/electronic products and automobile parts. Various methods for recycling ABS from waste including ABS and materials capable of minimizing damage to humans and the environment during the recycling process need to be explored.

### Technical Problem

The present specification provides a method for screening a solvent for extracting an acrylonitrile-butadiene-styrene copolymer, a method for recycling waste, a recycled acrylonitrile-butadiene-styrene copolymer, and a recycled acrylonitrile-butadiene-styrene copolymer composition.

### Technical Solution

An exemplary embodiment of the present specification provides a method for screening a solvent for extracting an acrylonitrile-butadiene-styrene copolymer. The method includes screening a solvent for extracting an acrylonitrile-butadiene-styrene copolymer from waste including the acrylonitrile-butadiene-styrene copolymer using the following Equation 1.

$$ABS solubility score = {x}_{1} {Log}_{10} Molecular Weight + {x}_{2} IAC\_Mean \qquad [ Equation 1 ]$$

In Equation 1, Molecular Weight is the molecular weight of the solvent,
IAC_Mean is the average information by an atomic number, and
x₁ and x₂ are each a real number from 0 to 5.

Another exemplary embodiment of the present specification provides a method for recycling waste including an acrylonitrile-butadiene-styrene copolymer. The method includes screening a solvent for extracting an acrylonitrile-butadiene-styrene copolymer using the above-described method for screening the solvent and extracting an acrylonitrile-butadiene-styrene copolymer from waste including the acrylonitrile-butadiene-styrene copolymer using the screened solvent.

Still another exemplary embodiment of the present specification provides a recycled acrylonitrile-butadiene-styrene copolymer extracted from waste including an acrylonitrile-butadiene-styrene copolymer using a solvent screened by the above-described method.

Yet another exemplary embodiment of the present specification provides a recycled acrylonitrile-butadiene-styrene copolymer composition including an acrylonitrile-butadiene-styrene copolymer extracted from waste including an acrylonitrile-butadiene-styrene copolymer, two or more good solvents of solvents with an ABS solubility score value of 5.5 or more according to the following Equation 1, and at least one non-solvent of solvents with an ABS solubility score value of less than 5.5 according to the following Equation 1.

$$ABS solubility score = {x}_{1} {Log}_{10} Molecular Weight + {x}_{2} IAC\_Mean \qquad [ Equation 1 ]$$

In Equation 1, Molecular Weight is the molecular weight of the solvent,
IAC_Mean is the average information by an atomic number, and
x₁ and x₂ are each a real number from 0 to 5.

### Advantageous Effects

According to an exemplary embodiment of the present specification, it is possible to find various solvents capable of being used in the process of extracting an acrylonitrile-butadiene-styrene copolymer from waste including an acrylonitrile-butadiene-styrene copolymer.

According to another exemplary embodiment of the present specification, it is possible to find various good solvent-non-solvent combinations suitable for the process of extracting an acrylonitrile-butadiene-styrene copolymer from waste including an acrylonitrile-butadiene-styrene copolymer.

### BRIEF DESCRIPTION OF DRAWINGS

FIGS. 1 and 2 are graphs showing ABS solubility score values calculated by Equation 1 according to the present specification.

### DETAILED DESCRIPTION

Hereinafter, the present specification will be described in detail.

The present specification has been made in an effort to provide a parameter capable of classifying and evaluating a solvent available for extracting an acrylonitrile-butadiene-styrene copolymer (ABS) from waste by predicting the solvent. Through this, a new solvent capable of extracting ABS may be discovered. Here, the solvent capable of extracting ABS means all solvents used for extracting ABS from waste, and specifically, may include not only a good solvent that has high solubility for ABS and thus can dissolve ABS from waste, but also a non-solvent that can obtain ABS by precipitating ABS from a good solvent in which ABS is dissolved. Further, one or more solvents can be mixed and used for one role. For example, a single solvent may be used as a good solvent, and a mixture of two or more solvents may be used as the good solvent, and in this case, in consideration of a mixing ratio of each solvent in a mixed solvent, that is, a weight fraction, an ABS solubility score value is calculated by substituting the weight fraction into Equation 1 to be described below.

An exemplary embodiment of the present specification provides a method for screening a solvent for extracting an acrylonitrile-butadiene-styrene copolymer, the method including screening a solvent for extracting an acrylonitrile-butadiene-styrene copolymer from waste including the acrylonitrile-butadiene-styrene copolymer using the following Equation 1.

$$ABS solubility score = {x}_{1} {Log}_{10} Molecular Weight + {x}_{2} IAC\_Mean \qquad [ Equation 1 ]$$

In Equation 1, Molecular Weight is the molecular weight of the solvent,
IAC_Mean is the average information by an atomic number, and
x₁ and x₂ are each a real number from 0 to 5.
   In an exemplary embodiment of the present specification, the affinity between ABS and a solvent is calculated using data such as the molecular weight of the solvent and the mean of information of atomic composition index, and based on the calculated result, it is possible to classify a good solvent that is mixed well with ABS, that is, a solvent capable of separating ABS from waste.

In an exemplary embodiment of the present specification, Equation 1 means a solubility score in consideration of factors capable of determining the solubility between ABS and a solvent along with the variables of experimental conditions such as temperature. The value of Equation 1 increases as the solubility for ABS increases.

In an exemplary embodiment of the present specification, through the value of Equation 1, a good solvent and a non-solvent may be distinguished based on a reference value for distinguishing the good solvent and the non-solvent of 4.75, 4.8, 4.9, 5, 5.1, 5.2, 5.3, 5.4, 5.5, 5.6, 5.7, 5.8, 5.9, or 6. Specifically, when the value is the reference value or more, the solvent may be determined as a good solvent, and when the value is less than the reference value, the solvent may be determined as a non-solvent.

In Equation 1 of an exemplary embodiment of the present specification, Molecular Weight is the molecular weight of the solvent. Since dissolving ABS from waste means that the solvent diffuses and permeates into the ABS whose chains are present while being entangled to disentangle and dissolve the chains of ABS, the effect of the molecular weight of the solvent on the diffusion of the solvent is reflected.

In Equation 1 of an exemplary embodiment of the present specification, IAC_Mean is the average information by an atomic number as the mean of information of atomic composition index, and expresses the interaction with the ABS polymer in consideration of the complexity of solvent molecules. In this case, since the interaction between the solvent and the ABS polymer changes depending on the ratio of styrene and acrylonitrile in the ABS polymer, IAC_Mean is a factor to consider the ratio of styrene and acrylonitrile in the ABS polymer.

In Equation 1 of an exemplary embodiment of the present specification, IAC_Mean may be obtained using a descriptor calculation program such as Dragon.

In an exemplary embodiment of the present specification, x₁ is a control variable to consider the effect of solvent molecular weight on the diffusion coefficient. x₁ is affected mainly by the process temperature, and for example, when the process temperature increases, the effect of the diffusion coefficient depending on molecular weight is weakened, so that the value of x₁ is also lowered, and in contrast, when the temperature decreases, the effect of the diffusion coefficient depending on molecular weight is significant, so that the value of x₁ is increased. Due to the control variable of x₁, there is an advantage in that it is possible to describe the phenomenon in which the solubility of ABS changes depending on the process temperature.

In an exemplary embodiment of the present specification, x₁ is a real number from 0 to 5, specifically a real number from 0 to 4, and more specifically a real number from 0 to 3, and may be preferably 2.

In an exemplary embodiment of the present specification, x₂ is a control variable determined by the interaction with the solvent according to the ratio of styrene and acrylonitrile in the ABS polymer. x₂ needs to be adjusted according to the ratio of styrene and acrylonitrile in the ABS polymer and the process conditions (temperature, pressure, and the like), and for example, when the content of acrylonitrile in the ABS polymer is increased, the interaction between the solvent and ABS is increased, so the value of x₂ is also increased.

In an exemplary embodiment of the present specification, x₂ is a real number from 0 to 5, specifically a real number from 0 to 4, and more specifically a real number from 0 to 3, and may be preferably 1.

In an exemplary embodiment of the present specification, a solvent with an ABS solubility score value of 5.5 or more according to Equation 1 may be determined to be a good solvent for an acrylonitrile-butadiene-styrene copolymer. In this case, a good solvent means a solvent capable of dissolving an acrylonitrile-butadiene-styrene copolymer, and is a solvent capable of dissolving and extracting the acrylonitrile-butadiene-styrene copolymer from waste.

In an exemplary embodiment of the present specification, when x₁ is 2 and x₂ is 1, a solvent with an ABS solubility score value of 5.5 or more according to Equation 1 may be determined to be a good solvent for an acrylonitrile-butadiene-styrene copolymer.

In an exemplary embodiment of the present specification, a solvent with an ABS solubility score value of less than 5.5 according to Equation 1 may be determined to be a non-solvent for an acrylonitrile-butadiene-styrene copolymer. In this case, a non-solvent means a solvent having a low solubility for an acrylonitrile-butadiene-styrene copolymer, and is a solvent that can be used when precipitating an acrylonitrile-butadiene-styrene copolymer from an extracted acrylonitrile-butadiene-styrene copolymer solution.

In an exemplary embodiment of the present specification, when x₁ is 2 and x₂ is 1, a solvent with an ABS solubility score value of less than 5.5 according to Equation 1 may be determined to be a non-solvent for an acrylonitrile-butadiene-styrene copolymer.

An exemplary embodiment of the present specification provides a method for screening a solvent for extracting an acrylonitrile-butadiene-styrene copolymer. The method includes selecting two or more of solvents with an ABS solubility score value of 5.5 or more according to Equation 1 as a good solvent for extracting an acrylonitrile-butadiene-styrene copolymer from waste including the acrylonitrile-butadiene-styrene copolymer. The method further includes selecting at least one of the solvents with an ABS solubility score value of less than 5.5 according to Equation 1 as a non-solvent for extracting an acrylonitrile-butadiene-styrene copolymer from waste including the acrylonitrile-butadiene-styrene copolymer.

In an exemplary embodiment of the present specification, a difference between the ABS solubility score value of the good solvent and the ABS solubility score value of the non-solvent may be 0.1 or more, 0.2 or more, 0.3 or more, 0.4 or more, 0.5 or more, 0.6 or more, 0.7 or more, 0.8 or more, 0.9 or more, 1 or more, 1.1 or more, 1.2 or more, 1.3 or more, 1.4 or more, 1.5 or more, 1.6 or more, 1.7 or more, 1.8 or more, 1.9 or more, 2 or more, 2.1 or more, 2.2 or more, 2.3 or more, 2.4 or more, 2.5 or more, 2.6 or more, 2.7 or more, 2.8 or more, 2.9 or more, 3 or more, 3.1 or more, 3.2 or more, 3.3 or more, 3.4 or more, 3.5 or more, 3.6 or more, 3.7 or more, 3.8 or more, 3.9 or more, 4 or more, 4.1 or more, 4.2 or more, 4.3 or more, 4.4 or more, 4.5 or more, 4.6 or more, 4.7 or more, 4.8 or more, 4.9 or more, 5 or more, 5.1 or more, 5.2 or more, 5.3 or more, 5.4 or more, 5.5 or more, 5.6 or more, 5.7 or more, 5.8 or more, 5.9 or more, or 6 or more.

In an exemplary embodiment of the present specification, the difference between the ABS solubility score value of the good solvent and the ABS solubility score value of the non-solvent may be 0.5 or more.

In an exemplary embodiment of the present specification, the good solvent and the non-solvent may each have a TD50 (Rat) of 15 mg/kg or more predicted by the Discovery Studio TOPKAT Module of BIOVIA. In this case, TD50 is a dose at which 50% of experimental animals die or produce undesirable toxic reactions when a test material is administered to experimental animals, and is usually expressed as mg per kg of body weight. Therefore, since lower TD50 values imply undesirable toxicity at small amounts of material, the material is more toxic. Conversely, a higher TD50 means that the material is relatively harmless because the material is not harmful to the body even though a large amount of material is ingested. TD50 values vary depending on experimental animals (for example, mouse, rat, and the like), and the TD50 values for rats as experimental animals were used in the present invention.

Another exemplary embodiment of the present specification provides a method for recycling waste including an acrylonitrile-butadiene-styrene copolymer, the method including screening a solvent for extracting an acrylonitrile-butadiene-styrene copolymer using the above-described method for screening the solvent, and extracting an acrylonitrile-butadiene-styrene copolymer from waste including the acrylonitrile-butadiene-styrene copolymer using the screened solvent.

In the present specification, the description of the screening step in the waste recycling method may reference the above-described method for screening a solvent for extracting ABS.

In an exemplary embodiment of the present specification, the extracting of the acrylonitrile-butadiene-styrene copolymer may include a dissolution process of bringing waste including the acrylonitrile-butadiene-styrene copolymer into contact with a good solvent with an ABS solubility score value of 5.5 or more according to Equation 1 and obtaining an acrylonitrile-butadiene-styrene copolymer solution by dissolving the acrylonitrile-butadiene-styrene copolymer in the waste in the good solvent.

In an exemplary embodiment of the present specification, the extracting of the acrylonitrile-butadiene-styrene copolymer may include: a dissolution process of bringing waste including the acrylonitrile-butadiene-styrene copolymer into contact with a good solvent with an ABS solubility score value of 5.5 or more according to Equation 1 and obtaining an acrylonitrile-butadiene-styrene copolymer solution by dissolving the acrylonitrile-butadiene-styrene copolymer in the waste in the good solvent, and a precipitation process of precipitating the acrylonitrile-butadiene-styrene copolymer from the solution by bringing the solution into contact with a non-solvent with an ABS solubility score value of less than 5.5 according to Equation 1.

In an exemplary embodiment of the present specification, the extracting of the acrylonitrile-butadiene-styrene copolymer may include: a dissolution process of bringing waste including the acrylonitrile-butadiene-styrene copolymer into contact with two or more good solvents of solvents with an ABS solubility score value of 5.5 or more according to Equation 1 and obtaining an acrylonitrile-butadiene-styrene copolymer solution by dissolving the acrylonitrile-butadiene-styrene copolymer in the waste in the good solvent, and a precipitation process of precipitating the acrylonitrile-butadiene-styrene copolymer from the solution by bringing the solution into contact with at least one non-solvent of solvents with an ABS solubility score value of less than 5.5 according to Equation 1.

Another exemplary embodiment of the present specification provides a method for recycling waste including an acrylonitrile-butadiene-styrene copolymer. The method includes selecting a good solvent and a non-solvent for extracting an acrylonitrile-butadiene-styrene copolymer using the above-described method for screening the solvent, bringing waste including the acrylonitrile-butadiene-styrene copolymer into contact with the two or more good solvents selected and obtaining an acrylonitrile-butadiene-styrene copolymer solution by dissolving the acrylonitrile-butadiene-styrene copolymer in the waste in the good solvent (hereinafter, referred to as a dissolution process), and precipitating the acrylonitrile-butadiene-styrene copolymer from the solution by bringing the solution into contact with the at least one non-solvent selected (hereinafter, referred to as a precipitation process).

In an exemplary embodiment of the present specification, the extracting of the acrylonitrile-butadiene-styrene copolymer may further include a separation process of separating a solid material from the acrylonitrile-butadiene-styrene copolymer solution obtained by the dissolution process. In this case, the solid material is a solid that is not dissolved in a good solvent of an acrylonitrile-butadiene-styrene copolymer, and corresponds to impurities.

In an exemplary embodiment of the present specification, the extracting of the acrylonitrile-butadiene-styrene copolymer may further include a rinsing process of rinsing the acrylonitrile-butadiene-styrene copolymer precipitated by the precipitation process.

In an exemplary embodiment of the present specification, the extracting of the acrylonitrile-butadiene-styrene copolymer may further include a drying process of drying the acrylonitrile-butadiene-styrene copolymer precipitated by the precipitation process.

Still another exemplary embodiment of the present specification provides a recycled acrylonitrile-butadiene-styrene copolymer extracted from waste including an acrylonitrile-butadiene-styrene copolymer using a solvent screened by the above-described method.

Yet another exemplary embodiment of the present specification provides a recycled acrylonitrile-butadiene-styrene copolymer composition including an acrylonitrile-butadiene-styrene copolymer extracted from waste including an acrylonitrile-butadiene-styrene copolymer, two or more good solvents of solvents with an ABS solubility score value of 5.5 or more according to the following Equation 1, and at least one non-solvent of solvents with an ABS solubility score value of less than 5.5 according to the following Equation 1.

$$ABS solubility score = {x}_{1} {Log}_{10} Molecular Weight + {x}_{2} IAC\_Mean \qquad [ Equation 1 ]$$

In Equation 1, Molecular Weight is the molecular weight of the solvent,
IAC_Mean is the average information by an atomic number, and
x₁ and x₂ are each a real number from 0 to 5.

Here, for the description on Equation 1, the above-described description is referenced to avoid duplication.

In an exemplary embodiment of the present specification, a recycled acrylonitrile-butadiene-styrene copolymer composition means a state in which an acrylonitrile-butadiene-styrene copolymer extracted by the good solvent from waste including an acrylonitrile-butadiene-styrene copolymer, a good solvent used during the extraction of the acrylonitrile-butadiene-styrene copolymer, and a non-solvent which separates and precipitates the extracted acrylonitrile-butadiene-styrene copolymer from the good solvent are all included. For the acrylonitrile-butadiene-styrene copolymer extracted depending on the ratio of the good solvent and the non-solvent, the acrylonitrile-butadiene-styrene copolymer dissolved in the good solvent and the precipitated acrylonitrile-butadiene-styrene copolymer may coexist or most of the acrylonitrile-butadiene-styrene copolymer may be precipitated and settled into a solid state.

In an exemplary embodiment of the present specification, when the weight of the good solvent in the recycled acrylonitrile-butadiene-styrene copolymer composition is set to 100, the content of the non-solvent is at least 100 parts by weight, and the solute may be precipitated by adding the non-solvent in an amount, which is equal to or more than the weight of the good solvent. The higher the content of non-solvent, the easier it is to precipitate the solute and the process time can be shortened, but in order to increase the purity in the solute, it is preferred to adjust the content of the non-solvent in consideration of the appropriate precipitation rate and the particle size of the precipitated solute.

In an exemplary embodiment of the present specification, when the weight of the good solvent is set to 100, the content of the non-solvent may be 100 parts by weight or more, 150 parts by weight or more, 200 parts by weight or more, 250 parts by weight or more, 300 parts by weight or more, 350 parts by weight or more, 400 parts by weight or more, 450 parts by weight or more or 500 parts by weight or more, 1000 parts by weight or less, 900 parts by weight or less, 800 parts by weight or less, 700 parts by weight or less, or 600 parts by weight or less.

In an exemplary embodiment of the present specification, the content of the good solvent is not particularly limited as long as the ABS in the waste can be dissolved. When the weight of the waste including the acrylonitrile-butadiene-styrene copolymer is set to 100, the content of the good solvent is at least 100 parts by weight, and the solute may be precipitated by adding the good solvent in an amount, which is equal to or more than the weight of the waste.

In an exemplary embodiment of the present specification, when the weight of the waste including the acrylonitrile-butadiene-styrene copolymer is set to 100, the content of the good solvent may be 100 parts by weight or more, 150 parts by weight or more, 200 parts by weight or more, 250 parts by weight or more, 300 parts by weight or more, 350 parts by weight or more, 400 parts by weight or more, 450 parts by weight or more or 500 parts by weight or more, 1000 parts by weight or less, 900 parts by weight or less, 800 parts by weight or less, 700 parts by weight or less, or 600 parts by weight or less.

In an exemplary embodiment of the present specification, the good solvent and the non-solvent are each independently any one of an alcohol-based solvent, a ketone-based solvent, an ester-based solvent, an ether-based solvent, a polar nonprotic solvent, a hydrocarbon solvent, and a halogen-containing solvent.

In an exemplary embodiment of the present specification, the good solvent is any one of an alcohol-based solvent, a ketone-based solvent, an ester-based solvent, an ether-based solvent, a polar nonprotic solvent, a hydrocarbon solvent, and a halogen-containing solvent.

In an exemplary embodiment of the present specification, the good solvent may include a main solvent present in an amount of 50 wt % or more in the good solvent; and an auxiliary solvent different from the main solvent and present in an amount of less than 50 wt % in the good solvent. Preferably, the weight ratio of the main solvent of the good solvent and the auxiliary solvent of the good solvent may be 50:50 to 99:1.

In an exemplary embodiment of the present specification, the main solvent of the good solvent basically means a solvent with a ratio of 50% or more in the entire good solvent, and has an advantage in that it is possible to include both a solvent in which interaction with a polymer is further considered when the polymer is dissolved and a solvent having a property of swelling the polymer well by mixing two or more solvents.

In an exemplary embodiment of the present specification, the good solvent in which the main solvent and the auxiliary solvent are mixed has an ABS solubility score value of 5.5 or more according to Equation 1 in consideration of the mixing ratio of each solvent in the mixed solvent, that is, the weight fraction, and is selected in consideration of the difference in solubility for between the non-solvent and acrylonitrile-butadiene-styrene copolymer used together.

In an exemplary embodiment of the present specification, a main solvent of the good solvent may be selected among dimethyl sulfoxide, n-methyl-2-pyrrolidinone, n,n-dimethylacetamide, Tetrahydrofuran (THF), 2-methylpyridine, 3,5-dimethylpyridine, acetylacetone, 1-methylimidazole, dimethyl isosorbide, gamma-valerolactone, 1,3-benzodioxole, diglyme, dichloromethane, chloroform, 1,1,2,2-tetrachloroethane, cyclopentanone, 2-cyclohexen-1-one, cyclohexanone, 2-pentanone, propanone, dimethylformamide, benzyl alcohol, carbon tetrachloride, 1,4-dioxane, trichloroethylene, or butanone, but is not limited thereto.

In an exemplary embodiment of the present specification, an auxiliary solvent of the good solvent may be selected among dimethyl sulfoxide, n-methyl-2-pyrrolidinone, n,n-dimethylacetamide, Tetrahydrofuran, 2-methylpyridine, 3,5-dimethylpyridine, acetylacetone, 1-methylimidazole, dimethyl isosorbide, gamma-valerolactone, 1,3-benzodioxole, diglyme, dichloromethane, chloroform, 1,1,2,2-tetrachloroethane, cyclopentanone, 2-cyclohexen-1-one, cyclohexanone, 2-pentanone, propanone, dimethylformamide, benzyl alcohol, carbon tetrachloride, 1,4-dioxane, trichloroethylene, butanone, oxalyl chloride, acetyl chloride, ethyl acetate, 3-methyl-2-butanone, 4-methylpyridine, acetyl acetate, 3-picoline, 2,3-pentanedione, morpholine, isopropyl acetate, pyrrole, 2-methylcyclopentanone, 2-methylpyrazine, cycloheptanone, 2,5-dimethylpyrazine, propanoic anhydride, 1,1-ethanediol diacetate, propionyl chloride, ethyl carbonochloridate, 1,1-dichloroethane, 3,6-dioxaoctane, 3,3-dimethyl-2-butanone, 3-methylcyclohexanone, 1,1-dichloro-2,2,2-trifluoroethane, chloroethyl chloroformate, methyl dichloroacetate, dichloroacetyl chloride, triethyl phosphate, dimethyl phthalate, chloroacetyl chloride, 3-chloropropanoyl chloride, methyl-5-(dimethylamino)-2-methyl-5-oxopentanoate, diethylene glycol diacetate, diethyl malate, dimethyl 1,4-cyclohexanedicarboxylate, diethyl adipate, diisopropyl methylphosphonate, dimethylcarbamoyl chloride, oxybis[chloromethane], isonicotinic acid ethyl ester, diethyl glutarate, ethylene sulfite, dimethyl adipate, dimethyl methylglutarate, ethyl succinate, n,n-diethyl-m-toluamide, 2-(2-ethoxyethoxy)ethyl acetate, ethyl chloroacetate, methoxyacetyl chloride, chloroacetic acid methyl ester, dimethyl glutarate, diethyl malonate, methyl carbonochloridate, methyl 2-hydroxybenzoate, 2-chlorophenol, ethyl oxalate, 2-acetyloxyethyl acetate, dimethyl succinate, 1,1,2-trichloroethane, 2-chloropyridine, 1-(2-hydroxyethyl)pyrrolidin-2-one, 2-[2-(diethylamino)ethoxy]ethanol, dimethyl malonate, 2,5-ditert-butylphenol, n-formylmorpholine, 4-morpholinepropanamine, 3,4-dihydrochromen-2-one, 3-methoxy-n,n-dimethylpropanamide, 4-(2-hydroxyethyl)morpholine, isopropyl acetoacetate, 2-acetylpyridine, 4-methoxyacetophenone, 3-methyl-2-oxazolidone, 1-ethoxy-2-(2-ethoxyethoxy)ethane, 2,6,6-trimethylcyclohex-2-ene-1,4-dione, 1,3-dimethyl-2-imidazolidinone, 3-methoxybutyl acetate, 2-isopropoxyethyl acetate, ethyl 3-ethoxypropionate, octahydrocoumarin, ethyl acetoacetate, 1-(2-hydroxyphenyl)ethanone, methyl benzoate, 1-(3-methoxypropoxy)propan-1-ol, n-sec-butylpyrrolidone, 2-methoxypropyl acetate, 2-ethoxyethanol acetate, 1-methoxy-2-propyl acetate, paraldehyde, tetramethylurea, methyl acetoacetate, 2-tert-butyl-4-methylphenol, 2-tert-butyl-5-methylphenol, 4-methoxyphenol, diethylene glycol monoethyl ether, 2-methoxyethyl acetate, diethyl carbonate, ethyl lactate, 1-tetralone, 2-tert-butylphenol, thymol, 3-tert-butylphenol, 2-methyl-5-propan-2-ylphenol, p-(sec-butyl)phenol, 1-ethylpyrrolidin-2-one, or 2-(2-methoxyethoxy)ethanol, but is not limited thereto.

In an exemplary embodiment of the present specification, the non-solvent is any one of an alcohol-based solvent, a ketone-based solvent, an ester-based solvent, an ether-based solvent, a polar nonprotic solvent, a hydrocarbon solvent, or a halogen-containing solvent.

In an exemplary embodiment of the present specification, the single non-solvent has an ABS solubility score value of less than 5.5 according to Equation 1, and is selected in consideration of the difference in solubility for between the good solvent and acrylonitrile-butadiene-styrene copolymer used together.

In an exemplary embodiment of the present specification, the single non-solvent may be selected among 2-methylfuran, 1-methyl-1H-pyrrole, thiophene, diethylene glycol monobutyl ether, 2-butoxyethanol, methanol, ethanol, formamide, 2-[(2-methylpropan-2-yl)oxymethyl]oxirane, methyl lactate, 1,3-dithiolane, n-ethyl-morpholine, 2-methylindole, 3-ethyl-5-methylphenol, 2,4,5-trimethylphenol, ethyl 2-methylpropanoate, sec-butyl acetate, propyl propionate, ethyl butyrate, 2-methylpropyl acetate, n-butyl acetate, acetophenone, benzofuran, 4-isopropylpyridine, 3-methoxy-3-methyl-1-butanol, 1-(dimethylamino)propan-2-ol, methyl isobutyrate, methyl butyrate, ethyl propionate, propyl acetate, pyrimidine, 2,6-dimethylpyridine, 2,5-dimethylpyridine, 2,4-dimethylpyridine, 4-ethylpyridine, 3,4-dimethylpyridine, ethylene glycol methyl ethyl ether, 2,2-dimethoxypropane, 3-methoxy-1-butanol, 2-(dimethylamino)-ethanol, 1,1-dimethoxyethane, 1-hydroxypropan-2-one, 4-methylpentan-2-one, 3-hexanone, 2-hexanone, tetrahydrothiophene, 2,3-dihydropyran, 2-methyltetrahydrofuran, toluene, trimethylphosphine, benzene, 2-propanol, acetonitrile, or 2-methyl-1-buten-3-yne, but is not limited thereto.

In an exemplary embodiment of the present specification, the non-solvent may include a main solvent present in an amount of 50 wt % or more in the non-solvent; and an auxiliary solvent different from the main solvent and present in an amount of less than 50 wt % in the non-solvent. Preferably, the weight ratio of the main solvent of the non-solvent and the auxiliary solvent of the non-solvent may be 50:50 to 99:1.

In an exemplary embodiment of the present specification, the main solvent of the non-solvent basically means a solvent with a ratio of 50% or more in the entire non-solvent, and has an advantage in that it is possible to include both a solvent in which interaction with a polymer is further considered when the polymer is dissolved and a solvent having a property of swelling well the polymer by mixing two or more solvents.

In an exemplary embodiment of the present specification, the non-solvent in which the main solvent and the auxiliary solvent are mixed has an ABS solubility score value of less than 5.5 according to Equation 1 in consideration of the mixing ratio of each solvent in the mixed solvent, that is, the weight fraction, and is selected in consideration of the difference in solubility for between the good solvent and acrylonitrile-butadiene-styrene copolymer used together.

In an exemplary embodiment of the present specification, the main solvent of the non-solvent and the auxiliary solvent of the non-solvent are different from each other, and may be each independently selected among 2-methylfuran, 1-methyl-1H-pyrrole, thiophene, diethylene glycol monobutyl ether, 2-butoxyethanol, methanol, ethanol, formamide, 2-[(2-methylpropan-2-yl)oxymethyl]oxirane, methyl lactate, 1,3-dithiolane, n-ethyl-morpholine, 2-methylindole, 3-ethyl-5-methylphenol, 2,4,5-trimethylphenol, ethyl 2-methylpropanoate, sec-butyl acetate, propyl propionate, ethyl butyrate, 2-methylpropyl acetate, n-butyl acetate, acetophenone, benzofuran, 4-isopropylpyridine, 3-methoxy-3-methyl-1-butanol, 1-(dimethylamino)propan-2-ol, methyl isobutyrate, methyl butyrate, ethyl propionate, propyl acetate, pyrimidine, 2,6-dimethylpyridine, 2,5-dimethylpyridine, 2,4-dimethylpyridine, 4-ethylpyridine, 3,4-dimethylpyridine, ethylene glycol methyl ethyl ether, 2,2-dimethoxypropane, 3-methoxy-1-butanol, 2-(dimethylamino)-ethanol, 1,1-dimethoxyethane, 1-hydroxypropan-2-one, 4-methylpentan-2-one, 3-hexanone, 2-hexanone, tetrahydrothiophene, 2,3-dihydropyran, 2-methyltetrahydrofuran, toluene, trimethylphosphine, benzene, 2-propanol, acetonitrile, or 2-methyl-1-buten-3-yne, but are not limited thereto.

Yet another exemplary embodiment of the present specification provides a recycled acrylonitrile-butadiene-styrene copolymer obtained by filtering the above-described composition.

In another exemplary embodiment of the present specification, for an acrylonitrile-butadiene-styrene copolymer extracted by the good solvent from waste including the acrylonitrile-butadiene-styrene copolymer in a waste recycled acrylonitrile-butadiene-styrene copolymer composition, most of the acrylonitrile-butadiene-styrene copolymer may be precipitated by the non-solvent and settled into a solid state. The recycled acrylonitrile-butadiene-styrene copolymer may be obtained by filtering the composition including the acrylonitrile-butadiene-styrene copolymer in the precipitated state.

In still another exemplary embodiment of the present specification, the recycled acrylonitrile-butadiene-styrene copolymer includes all the states in which the composition has been filtered and in which excess solvents have been removed by drying the composition after filtration.

In an exemplary embodiment of the present specification, the recycled acrylonitrile-butadiene-styrene copolymer includes an additive derived from waste. In this case, the additive derived from waste is not particularly limited as long as the additive is added for preparing the waste, and examples thereof include a flame retardant, and the like. Since an additive such as a flame retardant is already included when products are produced using the recycled acrylonitrile-butadiene-styrene copolymer, there is an advantage in that the additive such as a flame retardant is not added or the addition amount can be reduced.

In an exemplary embodiment of the present specification, the waste is not particularly limited as long as it includes ABS, but may be, for example, a waste home appliance including ABS, usually a refrigerator, a washing machine, a television set, and the like.

In an exemplary embodiment of the present specification, the recycled acrylonitrile-butadiene-styrene copolymer is ABS extracted by solvent extraction, and low-molecular weight ABSs are filtered out during solvent extraction, resulting in a higher average molecular weight than ABS prepared by polymerization or ABS in waste. Furthermore, as the average molecular weight of ABS is changed, dynamic physical properties affected by the average molecular weight such as the glass transition temperature (Tg) thereof are changed, so dynamic physical properties such as the glass transition temperature (Tg) are also increased as the average molecular weight of ABS is increased.

### EXAMPLES

Hereinafter, the present specification will be described in more detail through Examples. However, the following Examples are provided only for exemplifying the present specification, but are not intended to limit the present specification.

### EXAMPLES

### Example 1

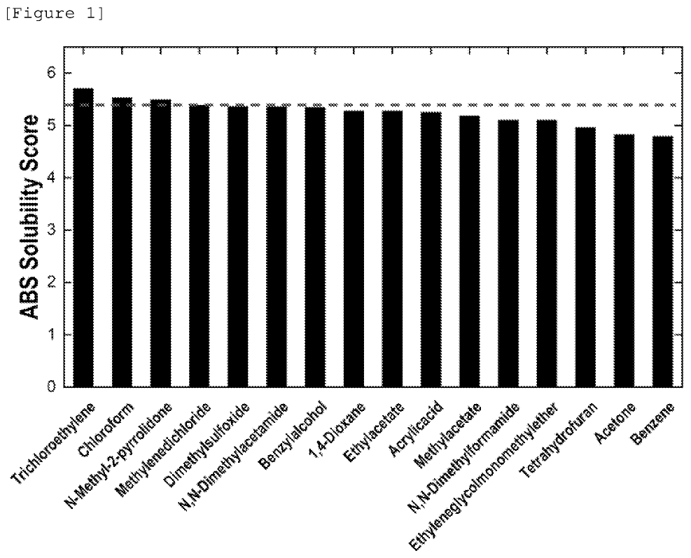

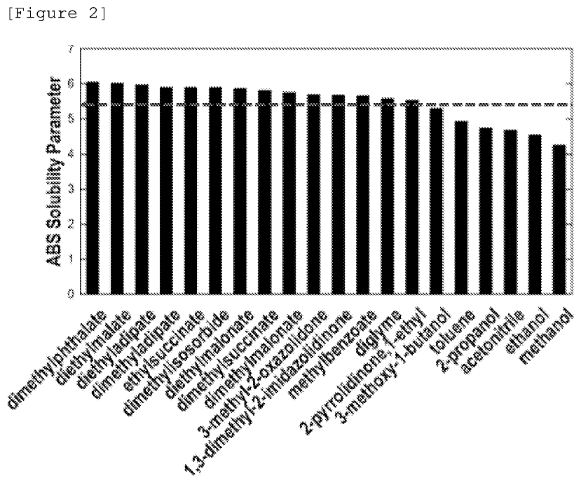

FIGS. 1 and 2 illustrate examples of ABS solubility in actual solvents based on literature values for the solubility of ABS in solvents. In this case, x₁ is 2, and x₂ is 1.

For x₁ and x₂ in the following Table 1, x₁ and x₂ were determined to be 2 and 1, respectively, in consideration of a temperature of 25° C. and a 4:1 ratio of styrene and acrylonitrile in ABS, in order to match the trend with the experimental values in the following Document #1 (Peng et al, J. Macromol. Sci. Part B, 2010, 49, 864).

The IAC_Mean value was extracted as a general-purpose descriptor calculation program such as Dragon based on the solvent molecular structure.

TABLE 1

| | Molecular | | ABS |
|---|---|---|---|
| Name | weight | IAC_Mean | solubility |
| 1,1-dichloro-2,2,2-trifluoroethane | 152.931 | 1.90563 | 6.274621 |
| Chloroethylchloroformate | 142.969 | 1.93625 | 6.246734 |
| Methyldichloroacetate | 142.969 | 1.93625 | 6.246734 |
| Dichloroacetylchloride | 147.388 | 1.84237 | 6.179294 |
| Triethylphosphate | 182.155 | 1.54224 | 6.063122 |
| Dimethylphthalate | 194.184 | 1.48335 | 6.059777 |
| Chloroacetylchloride | 112.943 | 1.95021 | 6.055929 |
| 3-chloropropanoylchloride | 126.969 | 1.84643 | 6.053825 |
| methyl-5-(dimethylamino)-2- | 187.236 | 1.48118 | 6.025959 |
| methyl-5-oxopentanoate | | | |
| Diethyleneglycoldiacetate | 190.194 | 1.46182 | 6.020214 |
| Diethylmalate | 190.194 | 1.46182 | 6.020214 |
| 1,4-cyclohexanedicarboxyl- | 200.232 | 1.39958 | 6.002647 |
| icaciddimethylester | | | |
| Diethyladipate | 202.248 | 1.36631 | 5.978078 |
| di-isopropylmethylphosphonate | 180.182 | 1.45402 | 5.965443 |
| Dimethylcarbamoylchloride | 107.539 | 1.89624 | 5.959372 |
| Oxybischloromethane | 114.959 | 1.83659 | 5.957676 |
| Isonicotinicacidethylester | 151.163 | 1.59546 | 5.954351 |
| 1,1,2,2-tetrachloroethane | 167.849 | 1.5 | 5.949838 |
| Diethylglutarate | 188.221 | 1.39145 | 5.940786 |
| Ethylenesulfite | 108.116 | 1.84643 | 5.91421 |
| Dimethyladipate | 174.194 | 1.41955 | 5.901616 |
| dimethyl2-methylglutarate | 174.194 | 1.41955 | 5.901616 |
| Ethylsuccinate | 174.194 | 1.41955 | 5.901616 |
| Dimethylisosorbide | 174.194 | 1.41955 | 5.901616 |
| n,n-diethyl-m-toluamide | 191.269 | 1.32495 | 5.888239 |
| 2-(2-ethoxyethoxy)-ethanolacetate | 176.21 | 1.37878 | 5.870841 |
| Ethylchloroacetate | 122.55 | 1.68939 | 5.866017 |
| methoxyacetylchloride | 108.524 | 1.78992 | 5.860972 |
| carbonochloridicacidethylester | 108.524 | 1.78992 | 5.860972 |
| chloroaceticacidmethylester | 108.524 | 1.78992 | 5.860972 |
| pentanedioicaciddimethylester | 160.168 | 1.4509 | 5.860052 |
| diethylmalonate | 160.168 | 1.4509 | 5.860052 |
| carbonochloridicacidmethylester | 94.497 | 1.90563 | 5.856466 |
| 2-hydroxy-benzoicacidmethylester | 152.147 | 1.47135 | 5.835877 |
| 2-chlorophenol | 128.556 | 1.61433 | 5.832515 |
| ethyloxalate | 146.141 | 1.48547 | 5.815014 |
| 1,1-ethanedioldiacetate | 146.141 | 1.48547 | 5.815014 |
| 1,2-ethanedioldiacetate | 146.141 | 1.48547 | 5.815014 |
| dimethylsuccinate | 146.141 | 1.48547 | 5.815014 |
| 1,1,2-trichloroethane | 133.404 | 1.56127 | 5.811608 |
| oxalylchloride | 126.926 | 1.58496 | 5.792061 |
| 2-chloropyridine | 113.545 | 1.67673 | 5.787066 |
| 1-(2-hydroxyethyl)-2-pyrrolidinone | 129.157 | 1.54375 | 5.765986 |
| 2-2-(diethylamino)ethoxyethanol | 161.242 | 1.34986 | 5.764816 |
| dimethylmalonate | 132.115 | 1.52218 | 5.764084 |
| 2,5-bis(1,1-dimethylethyl)phenol | 206.324 | 1.11727 | 5.746369 |
| n-formylmorpholine | 115.13 | 1.6087 | 5.731077 |
| 4-morpholinepropanamine | 144.215 | 1.40615 | 5.724171 |
| 3,4-dihydro-2H-1-benzopyran-2- | 148.159 | 1.37796 | 5.719416 |
| one | | | |
| 3-methoxy-n,n- | 131.173 | 1.4769 | 5.712589 |
| dimethylpropanamide | | | |
| 4-(2-hydroxyethyl)morpholine | 131.173 | 1.4769 | 5.712589 |
| 3-oxobutanoicacid-1- | 144.168 | 1.39461 | 5.712348 |
| methylethylester | | | |
| 2-acetylpyridine | 121.137 | 1.54356 | 5.710114 |
| 4-methoxyacetophenone | 150.174 | 1.35666 | 5.709849 |
| 3-methyl-2-oxazolidone | 101.104 | 1.68939 | 5.698927 |
| 1,1′-oxybis-2-ethoxy-ethane | 162.227 | 1.2782 | 5.698446 |
| 2,6,6-trimethylcyclohex-2-ene- | 152.19 | 1.32578 | 5.690552 |
| 1,4-dione | | | |
| 1,3-dimethyl-2-imidazolidinone | 114.146 | 1.56831 | 5.683231 |
| 3-methoxybutylacetate | 146.184 | 1.34707 | 5.67687 |
| 2-isopropoxyethylacetate | 146.184 | 1.34707 | 5.67687 |
| ethyl3hydroxyhexanoate | 146.184 | 1.34707 | 5.67687 |
| Octahydrocoumarin | 154.206 | 1.29056 | 5.666763 |
| propanoicacid anhydride | 130.142 | 1.43298 | 5.661815 |
| Ethylacetoacetate | 130.142 | 1.43298 | 5.661815 |
| 1-(2-hydroxyphenyl)ethanone | 136.148 | 1.39214 | 5.660163 |
| Methylbenzoate | 136.148 | 1.39214 | 5.660163 |
| gamma-valerolactone | 134.174 | 1.39958 | 5.654917 |
| Dipropyleneglycolmethylether | 148.2 | 1.30019 | 5.641886 |
| n-sec-butylpyrrolidone | 141.211 | 1.33972 | 5.639457 |
| Acetylchloride | 78.4976 | 1.84237 | 5.632083 |
| 2-methoxypropylacetate | 132.158 | 1.37878 | 5.620967 |
| 2-ethoxyethanolacetate | 132.158 | 1.37878 | 5.620967 |
| 1-methoxy-2-acetoxypropane | 132.158 | 1.37878 | 5.620967 |
| Paraldehyde | 132.158 | 1.37878 | 5.620967 |
| Tetramethylurea | 116.162 | 1.49046 | 5.620588 |
| Propionylchloride | 92.5242 | 1.68547 | 5.617981 |
| Methylacetoacetate | 116.115 | 1.47721 | 5.606987 |
| 1,3-benzodioxole | 122.121 | 1.42947 | 5.603051 |
| 2-(1,1-dimethylethyl)-4- | 164.244 | 1.16257 | 5.593549 |
| methylphenol | | | |
| 2-(1,1-dimethylethyl)-5- | 164.244 | 1.16257 | 5.593549 |
| methylphenol | | | |
| 4-methoxyphenol | 124.137 | 1.40208 | 5.589882 |
| Diglyme | 134.174 | 1.32496 | 5.580297 |
| diethyleneglycolmonoethylether | 134.174 | 1.32496 | 5.580297 |
| ethyleneglycolmono- | 118.131 | 1.41526 | 5.559988 |
| methyletheracetate | | | |
| Diethylcarbonate | 118.131 | 1.41526 | 5.559988 |
| Ethyllactate | 118.131 | 1.41526 | 5.559988 |
| 1-tetralone | 146.186 | 1.22857 | 5.558382 |
| Aceticanhydride | 102.089 | 1.52623 | 5.544188 |
| o-t-butylphenol | 150.218 | 1.18296 | 5.536404 |
| Thymol | 150.218 | 1.18296 | 5.536404 |
| 3-tert-butylphenol | 150.218 | 1.18296 | 5.536404 |
| 2-methyl-5-(1-methylethyl)phenol | 150.218 | 1.18296 | 5.536404 |
| p-(sec-butyl)phenol | 150.218 | 1.18296 | 5.536404 |
| 1-ethyl-2-pyrrolidinone | 113.158 | 1.42879 | 5.536161 |
| CHCl₃ | 119.378 | 1.37095 | 5.524799 |
| 2-(2-methoxyethoxy)ethanol | 120.147 | 1.35272 | 5.512146 |
| ((1,1- | 130.185 | 1.26467 | 5.493792 |
| dimethylethoxy)methyl)oxirane | | | |
| 2-hydroxy-propanoicacid- | 104.105 | 1.45656 | 5.491503 |
| methylester | | | |
| 1,1-dichloroethane | 98.9592 | 1.5 | 5.490912 |
| 1,3-dithiolane | 106.21 | 1.43537 | 5.487701 |
| n-ethyl-morpholine | 115.174 | 1.363 | 5.485709 |
| 2-methylindole | 131.174 | 1.24484 | 5.480536 |
| 3-ethyl-5-methylphenol | 136.191 | 1.20721 | 5.475507 |
| 2,4,5-trimethylphenol | 136.191 | 1.20721 | 5.475507 |
| 2-methylpropanoicacidethylester | 116.158 | 1.29546 | 5.425558 |
| sec-butylacetate | 116.158 | 1.29546 | 5.425558 |
| propylpropionate | 116.158 | 1.29546 | 5.425558 |
| Ethylbutyrate | 116.158 | 1.29546 | 5.425558 |
| aceticacid-2-methylpropylester | 116.158 | 1.29546 | 5.425558 |
| n-butylacetate | 116.158 | 1.29546 | 5.425558 |
| Acetophenone | 120.149 | 1.26393 | 5.42337 |
| Benzofuran | 118.133 | 1.2729 | 5.417642 |
| 2-methylpyrazine | 94.1145 | 1.46048 | 5.407793 |
| 4-(1-methylethyl)pyridine | 121.18 | 1.21924 | 5.386102 |
| 3,6-dioxaoctane | 118.174 | 1.24067 | 5.385714 |
| 3-methoxy-3-methylbutanol | 118.174 | 1.24067 | 5.385714 |
| 1-(dimethylamino)-2-propanol | 103.163 | 1.33616 | 5.363208 |
| 2-methylpropanoicacidmethylester | 102.132 | 1.33282 | 5.351144 |
| Methylbutyrate | 102.132 | 1.33282 | 5.351144 |
| ethylpropionate | 102.132 | 1.33282 | 5.351144 |
| isopropylacetate | 102.132 | 1.33282 | 5.351144 |
| n-propylacetate | 102.132 | 1.33282 | 5.351144 |
| Pyrimidine | 80.088 | 1.52192 | 5.329055 |
| 2,6-dimethylpyridine | 107.153 | 1.25329 | 5.313299 |
| 2,5-dimethylpyridine | 107.153 | 1.25329 | 5.313299 |
| 2,4-dimethylpyridine | 107.153 | 1.25329 | 5.313299 |
| 4-ethylpyridine | 107.153 | 1.25329 | 5.313299 |
| 3,5-dimethylpyridine | 107.153 | 1.25329 | 5.313299 |
| 3,4-dimethylpyridine | 107.153 | 1.25329 | 5.313299 |
| Ethyleneglycolmethylethylether | 104.148 | 1.26744 | 5.302742 |
| 2,2-dimethoxypropane | 104.148 | 1.26744 | 5.302742 |
| 3-methoxy-1-butanol | 104.148 | 1.26744 | 5.302742 |
| 1-methylimidazol | 82.1038 | 1.45914 | 5.287867 |
| 2-(dimethylamino)-ethanol | 89.1362 | 1.37841 | 5.278518 |
| Thiophene | 84.1396 | 1.39214 | 5.242141 |
| 2-methylpyridine | 93.1265 | 1.29583 | 5.233977 |
| 1,1-dimethoxyethane | 90.121 | 1.29879 | 5.208442 |
| 1-hydroxy-2-propanone | 74.0785 | 1.43537 | 5.174754 |
| 4-methyl-2-pentanone | 100.159 | 1.16743 | 5.16881 |
| 3-hexanone | 100.159 | 1.16743 | 5.16881 |
| 3,3-dimethyl-2-butanone | 100.159 | 1.16743 | 5.16881 |
| 2-hexanone | 100.159 | 1.16743 | 5.16881 |
| 2-methylfuran | 82.1005 | 1.32501 | 5.153702 |
| Tetrahydrothiophene | 88.1713 | 1.2389 | 5.129554 |
| 2,3-dihydropyran | 84.1164 | 1.2638 | 5.113561 |
| 2-methyltetrahydrofuran | 86.1323 | 1.19819 | 5.068522 |
| Toluene | 92.1384 | 0.99679 | 4.925671 |
| Trimethylphosphine | 76.0773 | 1.14011 | 4.90262 |
| Benzene | 78.1118 | 1 | 4.785433 |
| 2-propanol | 60.095 | 1.18872 | 4.746397 |
| Acetonitrile | 41.0519 | 1.45914 | 4.685807 |
| 2-methyl-1-buten-3-yne | 66.1011 | 0.99403 | 4.634447 |
| Ethanol | 46.0684 | 1.22439 | 4.551196 |
| Methanol | 32.0419 | 1.25162 | 4.263057 |

Among the solvents in Table 1, the ABS solubility score values of Equation 1 and the solvent classifications of the following Document #1 are compared in the following Table 2 for the solvents tested for dissolution of ABS in Document #1.

TABLE 2

| Solvent | ABS solubility score | Literature value |
|---|---|---|
| Methanol | 4.263057 | #1 Bad |
| Ethanol | 4.551196 | #1 Bad |
| Acetonitrile | 4.685807 | #1 Bad |
| 2-Propanol | 4.746397 | #1 Bad |
| Acetone | 4.823499 | #1 Good |
| Toluene | 4.925671 | #1 Good |
| Tetrahydrofuran | 4.954839 | #1 Good |
| N,N-Dimethylformamide | 5.351839 | #1 Good |
| 1,4-Dioxane | 5.268782 | #1 Good |
| Ethylacetate | 5.268782 | #1 Good |
| Benzylaocohol | 5.339736 | #1 Good |
| N,N-Dimethylacetamide | 5.351839 | #1 Good |
| Dimethylsulfoxide | 5.356623 | #1 Good |
| Methylenedichloride | 5.380068 | #1 Good |
| N-Methyl-2-pyrrolidone | 5.483729 | #1 Good |
| Chloroform | 5.524798 | #1 Good |
| Trichloroethylene | 5.696251 | #1 Good |

In Document #1, a 0.1% polymer was added to each solvent at room temperature, and then when the polymer was not dissolved after 8 hours, the polymer was labeled as bad, and when the polymer was dissolved, the polymer was labeled as good. Based on this, solvents were classified by confirmation with a Hansen solubility parameter (HSP). However, the Hansen solubility parameter is a parameter that roughly describes the thermodynamic solubility, and does not consider the diffusion of solvent molecules that need to be considered in the solubility of polymers.

Conversely, the parameters of Equation 1 of the present specification are parameters considering the molecular weight of the solvent and the complexity of the solvent molecules, and are more reliable than the Hansen solubility parameters.

As the value of Equation 1 in the present specification increases, the solubility increases, and in Table 2, it can be confirmed that the tendency of the value of Equation 1 to be increased reflects the tendency of ABS to be actually dissolved well.

In this case, the experiment in Document #1 was performed at 25° C., and the ABS solubility scores in Table 1 were also calculated at the same temperature.

### Experimental Example

A solution was prepared by adding an ABS waste sample to the good solvent shown in the following Table 3 and stirring the resulting mixture at 120 rpm at room temperature for 2 hours. Thereafter, ABS was precipitated by dropping a non-solvent shown in the following Table 3 into the solution. In this case, the weight ratio of waste ABS:good solvent:non-solvent is 1:10:10, and the weight ratio of main solvent and auxiliary solvent in the good solvent is 60:40.

After all non-solvents were dropped, a filtered solid was obtained by filtering the reaction solution with a filter. The filtered solid was further washed with the same non-solvent as the above non-solvent and dried to obtain recycled ABS.

TABLE 3

| | Good solvent | | | | Recovery |
|---|---|---|---|---|---|
| | Main | Auxiliary | Non- | Recovery | rate |
| | solvent | solvent | solvent | (g) | (%) |
| Experimental | Benzyl | dichloro- | 2-propanol | 5.05 | 63.125 |
| Example 1 | alcohol | methane | | | |
| Experimental | Benzyl | THF | 2-propanol | 3.97 | 49.625 |
| Example 2 | alcohol | | | | |
| Experimental | Dichloro- | THF | 2-propanol | 6.1 | 76.25 |
| Example 3 | methane | | | | |

ABS solubility score values for the solvents used in Table 3 are shown in the following Table 4.

Referring to Table 3 based on the ABS solubility score values in the following Table 4, dichloromethane has the highest ABS solubility, followed by THF and benzyl alcohol. Therefore, Experimental Example 3, in which THF was mixed while using dichloromethane as the main solvent, had the highest recovery rate, Experimental Example 1, in which dichloromethane was used as the auxiliary solvent, had the second highest recovery rate, and Experimental Example 2, in which benzyl alcohol and THF were mixed, showed the lowest recovery rate.

TABLE 4

| | ABS solubility score value | Classification |
|---|---|---|
| Benzyl alcohol | 5.88 | Good solvent |
| Dichloromethane | 6.24 | Good solvent |
| THF | 6.01 | Good solvent |
| 2-propanol | 4.75 | Non-solvent |

1. A method for screening a solvent for extracting an acrylonitrile-butadiene-styrene copolymer, comprising:
screening a solvent for extracting an acrylonitrile-butadiene-styrene copolymer from a waste including the acrylonitrile-butadiene-styrene copolymer using the following Equation 1:
$$ABS solubility score = {x}_{1} {Log}_{10} Molecular Weight + {x}_{2} IAC\_Mean \qquad [ Equation 1 ]$$
wherein Molecular Weight is a molecular weight of the solvent,
IAC_Mean is an average information by an atomic number, and
x₁ and x₂ are each a real number from 0 to 5.

2. The method of claim 1, further comprising:
selecting two or more solvents with a first ABS solubility score value of 5.5 or more according to Equation 1 as a good solvent for the acrylonitrile-butadiene-styrene copolymer; and
selecting at least one solvents with a second ABS solubility score value of less than 5.5 according to Equation 1 as a non-solvent for the acrylonitrile-butadiene-styrene copolymer.

3. The method of claim 2, wherein a difference between the first ABS solubility score value of the good solvent and the second ABS solubility score value of the non-solvent is 0.5 or more.

4. The method of claim 2, wherein the good solvent and the non-solvent each have a TD50 for rats of 15 mg/kg or more calculated by the Discovery Studio TOPKAT Module of BIOVIA.

5. The method of claim 1, wherein x₁ is 2 and x₂ is 1.

6. A method for recycling the waste comprising the acrylonitrile-butadiene-styrene copolymer, comprising:
screening the solvent for extracting the acrylonitrile-butadiene-styrene copolymer using the method claim 1; and
extracting the acrylonitrile-butadiene-styrene copolymer from the waste using the screened solvent.

7. The method of claim 6, wherein the extracting of the acrylonitrile-butadiene-styrene copolymer comprises:
a dissolution process of bringing the waste including the acrylonitrile-butadiene-styrene copolymer into contact with two or more good solvents of solvents with the first ABS solubility score value of 5.5 or more according to Equation 1 and obtaining an acrylonitrile-butadiene-styrene copolymer solution by dissolving the acrylonitrile-butadiene-styrene copolymer in the waste in the good solvent; and
a precipitation process of precipitating the acrylonitrile-butadiene-styrene copolymer from the acrylonitrile-butadiene-styrene copolymer solution by bringing the acrylonitrile-butadiene-styrene copolymer solution into contact with at least one non-solvent of solvents with the second ABS solubility score value of less than 5.5 according to Equation 1.

8. The method of claim 7, further comprising a separation process of separating a solid material from the acrylonitrile-butadiene-styrene copolymer solution obtained by the dissolution process.

9. The method of claim 7, further comprising a rinsing process of rinsing the acrylonitrile-butadiene-styrene copolymer precipitated by the precipitation process.

10. A recycled acrylonitrile-butadiene-styrene copolymer extracted from the waste comprising an acrylonitrile-butadiene-styrene copolymer using the solvent screened by the method of claim 1.

11. The recycled acrylonitrile-butadiene-styrene copolymer of claim 10, comprising an additive derived from the waste.

12. A recycled acrylonitrile-butadiene-styrene copolymer composition comprising:
an acrylonitrile-butadiene-styrene copolymer extracted from a waste comprising the acrylonitrile-butadiene-styrene copolymer;
two or more good solvents with a first ABS solubility score value of 5.5 or more according to the following Equation 1; and
at least one non-solvent with a second ABS solubility score value of less than 5.5 according to the following Equation 1:
$$ABS solubility score = {x}_{1} {Log}_{10} Molecular Weight + {x}_{2} IAC\_Mean \qquad [ Equation 1 ]$$
wherein Molecular Weight is a molecular weight of the solvent,
IAC_Mean is an average information by an atomic number, and
x₁ and x₂ are each a real number from 0 to 5.

13. The recycled acrylonitrile-butadiene-styrene copolymer composition of claim 12, wherein a difference between the first ABS solubility score value of the good solvent and the second ABS solubility score value of the non-solvent is 0.5 or more.

14. The recycled acrylonitrile-butadiene-styrene copolymer composition of claim 12, wherein the good solvent and the non-solvent each have a TD50 for rats of 15 mg/kg or more calculated by the Discovery Studio TOPKAT Module of BIOVIA.

15. The recycled acrylonitrile-butadiene-styrene copolymer composition of claim 12, wherein the good solvent and the non-solvent each independently include any one of an alcohol-based solvent, a ketone-based solvent, an ester-based solvent, an ether-based solvent, a polar nonprotic solvent, a hydrocarbon solvent, or a halogen-containing solvent.

16. The recycled acrylonitrile-butadiene-styrene copolymer composition of claim 12, wherein the good solvent comprises a main solvent in an amount of 50 wt % or more and an auxiliary solvent different from the main solvent in an amount of less than 50 wt %.

17. The recycled acrylonitrile-butadiene-styrene copolymer composition of claim 16, wherein the main solvent of the good solvent comprises one or more of dimethyl sulfoxide, n-methyl-2-pyrrolidinone, n,n-dimethylacetamide, Tetrahydrofuran, 2-methylpyridine, 3,5-dimethylpyridine, acetylacetone, 1-methylimidazole, dimethyl isosorbide, gamma-valerolactone, 1,3-benzodioxole, diglyme, dichloromethane, chloroform, 1,1,2,2-tetrachloroethane, cyclopentanone, 2-cyclohexen-1-one, cyclohexanone, 2-pentanone, propanone, dimethylformamide, benzyl alcohol, carbon tetrachloride, 1,4-dioxane, trichloroethylene, or butanone.

18. The recycled acrylonitrile-butadiene-styrene copolymer composition of claim 16, wherein the auxiliary solvent of the good solvent comprises one or more of dimethyl sulfoxide, n-methyl-2-pyrrolidinone, n,n-dimethylacetamide, Tetrahydrofuran, 2-methylpyridine, 3,5-dimethylpyridine, acetylacetone, 1-methylimidazole, dimethyl isosorbide, gamma-valerolactone, 1,3-benzodioxole, diglyme, dichloromethane, chloroform, 1,1,2,2-tetrachloroethane, cyclopentanone, 2-cyclohexen-1-one, cyclohexanone, 2-pentanone, propanone, dimethylformamide, benzyl alcohol, carbon tetrachloride, 1,4-dioxane, trichloroethylene, butanone, oxalyl chloride, acetyl chloride, ethyl acetate, 3-methyl-2-butanone, 4-methylpyridine, acetyl acetate, 3-picoline, 2,3-pentanedione, morpholine, isopropyl acetate, pyrrole, 2-methylcyclopentanone, 2-methylpyrazine, cycloheptanone, 2,5-dimethylpyrazine, propanoic anhydride, 1,1-ethanediol diacetate, propionyl chloride, ethyl carbonochloridate, 1,1-dichloroethane, 3,6-dioxaoctane, 3,3-dimethyl-2-butanone, 3-methylcyclohexanone, 1,1-dichloro-2,2,2-trifluoroethane, chloroethyl chloroformate, methyl dichloroacetate, dichloroacetyl chloride, triethyl phosphate, dimethyl phthalate, chloroacetyl chloride, 3-chloropropanoyl chloride, methyl-5-(dimethylamino)-2-methyl-5-oxopentanoate, diethylene glycol diacetate, diethyl malate, dimethyl 1,4-cyclohexanedicarboxylate, diethyl adipate, diisopropyl methylphosphonate, dimethylcarbamoyl chloride, oxybis[chloromethane], isonicotinic acid ethyl ester, diethyl glutarate, ethylene sulfite, dimethyl adipate, dimethyl methylglutarate, ethyl succinate, n,n-diethyl-m-toluamide, 2-(2-ethoxyethoxy)ethyl acetate, ethyl chloroacetate, methoxyacetyl chloride, chloroacetic acid methyl ester, dimethyl glutarate, diethyl malonate, methyl carbonochloridate, methyl 2-hydroxybenzoate, 2-chlorophenol, ethyl oxalate, 2-acetyloxyethyl acetate, dimethyl succinate, 1,1,2-trichloroethane, 2-chloropyridine, 1-(2-hydroxyethyl)pyrrolidin-2-one, 2-[2-(diethylamino)ethoxy]ethanol, dimethyl malonate, 2,5-ditert-butylphenol, n-formylmorpholine, 4-morpholinepropanamine, 3,4-dihydrochromen-2-one, 3-methoxy-n,n-dimethylpropanamide, 4-(2-hydroxyethyl)morpholine, isopropyl acetoacetate, 2-acetylpyridine, 4-methoxyacetophenone, 3-methyl-2-oxazolidone, 1-ethoxy-2-(2-ethoxyethoxy)ethane, 2,6,6-trimethylcyclohex-2-ene-1,4-dione, 1,3-dimethyl-2-imidazolidinone, 3-methoxybutyl acetate, 2-isopropoxyethyl acetate, ethyl 3-ethoxypropionate, octahydrocoumarin, ethyl acetoacetate, 1-(2-hydroxyphenyl)ethanone, methyl benzoate, 1-(3-methoxypropoxy)propan-1-ol, n-sec-butylpyrrolidone, 2-methoxypropyl acetate, 2-ethoxyethanol acetate, 1-methoxy-2-propyl acetate, paraldehyde, tetramethylurea, methyl acetoacetate, 2-tert-butyl-4-methylphenol, 2-tert-butyl-5-methylphenol, 4-methoxyphenol, diethylene glycol monoethyl ether, 2-methoxyethyl acetate, diethyl carbonate, ethyl lactate, 1-tetralone, 2-tert-butylphenol, thymol, 3-tert-butylphenol, 2-methyl-5-propan-2-ylphenol, p-(sec-butyl)phenol, 1-ethylpyrrolidin-2-one, or 2-(2-methoxyethoxy)ethanol.

19. The recycled acrylonitrile-butadiene-styrene copolymer composition of claim 12, wherein the at least one non-solvent comprises one or more of 2-methylfuran, 1-methyl-1H-pyrrole, thiophene, diethylene glycol monobutyl ether, 2-butoxyethanol, methanol, ethanol, formamide, 2-[(2-methylpropan-2-yl)oxymethyl]oxirane, methyl lactate, 1,3-dithiolane, n-ethyl-morpholine, 2-methylindole, 3-ethyl-5-methylphenol, 2,4,5-trimethylphenol, ethyl 2-methylpropanoate, sec-butyl acetate, propyl propionate, ethyl butyrate, 2-methylpropyl acetate, n-butyl acetate, acetophenone, benzofuran, 4-isopropylpyridine, 3-methoxy-3-methyl-1-butanol, 1-(dimethylamino)propan-2-ol, methyl isobutyrate, methyl butyrate, ethyl propionate, propyl acetate, pyrimidine, 2,6-dimethylpyridine, 2,5-dimethylpyridine, 2,4-dimethylpyridine, 4-ethylpyridine, 3,4-dimethylpyridine, ethylene glycol methyl ethyl ether, 2,2-dimethoxypropane, 3-methoxy-1-butanol, 2-(dimethylamino)-ethanol, 1,1-dimethoxyethane, 1-hydroxypropan-2-one, 4-methylpentan-2-one, 3-hexanone, 2-hexanone, tetrahydrothiophene, 2,3-dihydropyran, 2-methyltetrahydrofuran, toluene, trimethylphosphine, benzene, 2-propanol, acetonitrile, or 2-methyl-1-buten-3-yne.
